(19) United States
(12) Patent Application Publication
(10) Pub. No.: US 2020/0383593 A1
(43) Pub. Date: Dec. 10, 2020

(54) Device for Measuring the Mechanical Properties of Vocal Cords

(71) Applicant: Board of Regents, The University of Texas System, Austin, TX (US)

(72) Inventors: Vangelina Osteguin, San Antonio, TX (US); Melanie Foster, San Antonio, TX (US); Natalia Rafiq, Austin, TX (US); Divya Kamnani, Sugar Land, TX (US); Teja Guda, San Antonio, TX (US); Gregory Robert Dion, Schertz, TX (US)

(21) Appl. No.: 16/864,141

(22) Filed: Apr. 30, 2020

Related U.S. Application Data
(60) Provisional application No. 62/841,192, filed on Apr. 30, 2019.

(51) Int. Cl. A61B 5/03; G01N 3/42; G01N 3/06

(52) U.S. Cl. CPC A61B 5/036; G01N 3/42; G01N 3/066; A61B 5/08; A61B 2562/0247; A61B 2560/0431; A61B 2560/0418

(57) ABSTRACT

The invention provides a nanoindentation pen in the form of a device for measurement of mechanical properties of a tissue in a subject, the device comprising a dumbbell comprising an elongated shaft, the shaft being affixed at a first end to a pen tip for contacting the tissue, the shaft being affixed at a second opposite end to a terminal member, a linear actuator, and one or more pressure sensors that are in physical communication with the terminal member, the pressure sensor(s) generating a signal that indicates a force exerted on the terminal member by the pen tip via the shaft. In some embodiments, the inventive device uses statics and mechanics to calculate the stiffness of a human vocal fold based on a known displacement and a measured force. The pen tip is able to reach most sides of the vocal fold, making it ideal for all types of examination purposes.

### CROSS-REFERENCE TO RELATED APPLICATIONS

This application claims the benefit of U.S. Provisional Appl. No. 62/841,192, filed Apr. 30, 2019, the contents of which are incorporated by reference herein.

### FIELD OF THE INVENTION

The present invention generally relates to medicine and medical devices, in particular to a device for measuring the mechanical properties of vocal cords.

### BACKGROUND OF THE INVENTION

Damage to the vocal folds, also called vocal cords, can occur in various ways. Risk of laryngeal nerve damage can result from thyroid surgeries, head or neck cancers, or human papillomavirus (HPV). Additionally, vocal cord paralysis can be caused by neck or chest injury, stroke, noncancerous as well as cancerous tumors, infections, or neurological conditions such as multiple sclerosis or Parkinson's disease. Vocal fold damage may also occur in people who exert their voices, such as singers, teachers, and doctors, and is more likely to happen amongst smokers or the elderly. In general, vocal fold damage is a common complication of various ailments and conditions that is not limited to any particular age group or demographic.

There are various avenues for treating damage to the vocal folds. Steroids, botox, implants, stem cell treatment, and surgeries are all mechanisms for treating damage to the vocal folds. An ear, nose, and throat (ENT) physician may choose a treatment based on his or her experience, preference, or the particular situation. While all of the above treatments are currently being used, there is presently no means of quantifying which is most effective or even of monitoring the progress of the treatment. Currently, ENTs monitor progress through the sole basis of visual and audio inspection of the vocal folds. To examine vocal folds, a doctor might place a rigid laryngoscope or rigid endoscope for videostroboscopy in a patient's mouth, or a flexible endoscope can be passed through the patient's nose. These methods as well as calculating the frequency of the patient's voice have given physicians some tools to work with. However, the available techniques are still largely indirect in that they rely on visual inspection and do not provide any direct quantification of the mechanical properties of the vocal folds.

Vocal fold augmentation is a common procedure that affects all demographics. About 7.6% of the adult population suffers from speech related problems. Although there are several modes of treatments (steroids, botox, surgery, etc.), there is no means of quantifying which is most effective or of accurately monitoring the progress of the treatment. Ear, nose, and throat doctors (ENTs) currently monitor progress through visual and audio inspection of the vocal folds, which is largely ineffective with respect to the gathering of quantitative data that might inform treatment decisions.

Currently, ex vivo studies for understanding the mechanical properties of the vocal folds are done through nanoindentation, a common hardness test used for small volumes. Nanoindentation can either be force controlled or displacement controlled. One of the two variables is known and induced on the desired section of vocal fold area while the other variable is measured. This technique has been used to measure the stiffness of a material by examining the linear region of the load vs. displacement graph. This method utilizes bulky equipment and is unsuitable for in vivo use in patient examinations.

There is a need in the art for a device for the in vivo measurement of the mechanical properties of various tissues, where the device is small in size, flexible, durable, portable and has a simple and useful user interface. Such a device would find application in the research sector where laboratories have a need to develop improved treatments and to quantify disease progression and treatment efficacy.

### SUMMARY OF THE INVENTION

It is to be understood that both the foregoing general description of the embodiments and the following detailed description are exemplary, and thus do not restrict the scope of the embodiments.

In one aspect, the invention provides a device capable of detecting the differences in the mechanical properties of underlying tissues, comprising a small dumbbell tip acting as an indenter tip that is housed at the end of a long rigid tube or rod. In some embodiments, a linear actuator may be used to set a constant distance for deflection of the tissue during each test, and capacitive sensors touch the dumbell to measure the force of the tissue resisting movement.

In some embodiments, the dumbbell can have two spheres at its ends; a large one touching the capacitive force sensors placed within the tube and a much smaller one free to be in contact with the tissue. The displacement induced will be done through the linear actuator.

In some aspects, the force can be measured through four capacitive sensors. Three sensors can be placed equidistant from each other on the sides of the dumbbell, while the fourth sensor can be placed at the top. Placement of the sensors in this way can be such that they define a resultant force vector in 3D space that coincides with the direction of a displacement the tissue undergoes.

Since the displacement is known and the real force has been measured, a force vs. displacement graph can be plotted, and, from the linear region of this graph, the stiffness of the vocal fold can be measured and displayed to the user.

In some embodiments, the present invention is a device for measurement of mechanical properties of a tissue in a subject, the device comprising a dumbbell comprising an elongated shaft, the shaft being affixed at a first end to a pen tip for contacting the tissue, the shaft being affixed at a second opposite end to a terminal member; a linear actuator; and one or more pressure sensors that are in physical communication with the terminal member, the pressure sensor(s) generating a signal that indicates a force exerted on the terminal member by the pen tip via the shaft.

In some embodiments, the device of the present invention includes a linear actuator that can be configured to reliably and repeatedly move the shaft axially over a constant distance.

In some embodiments, the pen tip of the device of the present invention can be conical in shape, an apex of the cone being oriented to contact the tissue when the device is operated.

In some embodiments, one or both of the pen tip and the terminal member of the device of the present invention are spherical.

In some embodiments, a radius of the terminal member can be much larger than a radius of the pen tip.

In some embodiments, the shaft can have a cylindrical shape.

In some embodiments, the one or more pressure sensors can be capacitive pressure sensors.

In some embodiments, the device of the present invention can comprise four pressure sensors, wherein three pressure sensors are distributed at regular intervals radially about the shaft and one pressure sensor is in line with an axis of the shaft.

In some embodiments, the mechanical properties to be measured include tissue stiffness.

In some embodiments, the signal generated by the inventive device is an electrical signal.

In some embodiments, the inventive device further comprises circuitry for calculating a tissue stiffness from the electrical signal.

In some embodiments, the inventive device further comprises a dumbbell shell that encloses all or part of the dumbbell.

In certain embodiments of the present invention, the subject can be a human subject.

In certain embodiments, the human tissue in which the inventive device measures mechanical properties is vocal folds.

In certain embodiments, a length of the device is about 26 cm.

In certain embodiments, the device is cylindrical in shape overall and has a diameter of 1 cm or less.

In certain embodiments, the device is used in vivo.

In certain embodiments, the device is used ex vivo.

In certain embodiments, the pen tip, the terminal member and the shaft of the inventive device are all formed of stainless steel.

In certain embodiments, the pen tip, the terminal member and the shaft can be continuous.

In some embodiments, the inventive device is portable.

In some embodiments, the inventive device has a total weight of less than three pounds.

In some embodiments, the inventive device further comprises a display that displays a tissue stiffness quantity to a user.

In certain embodiments, the present invention can comprise a method for measuring stiffness of a biological tissue, the method comprising providing a dumbbell, the dumbbell having an elongated shape and comprising a pen tip at a first end, a terminal member at a second end and a shaft connecting the pen tip and the terminal member; contacting the pen tip with the biological tissue; actuating a linear actuator such that the pen tip displaces a portion of the biological tissue; and measuring a force exerted on the terminal member by the biological tissue via the pen tip and the shaft.

In some embodiments, the biological tissue of the method of the present invention can be human biological tissue.

In certain embodiments, the human biological tissue of the method of the present invention can be vocal folds.

In some embodiments, the method of the present invention can be performed in vivo.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description

### BRIEF DESCRIPTION OF THE FIGURES

FIG. 1 shows an overview of an embodiment of the inventive device.

FIG. 2 shows the four force vector components that act upon the dumbbell, the vector sum being oriented in a direction along the shaft of the dumbbell.

FIG. 3 shows a force vs. displacement curve of a kind that might be generated by an embodiment of the inventive device.

FIG. 4 shows a dumbbell resting inside a dumbbell shell in one embodiment of the inventive device.

FIG. 5 shows the component parts of one embodiment of a nanoindentation pen device in an exploded view of the present invention.

### DETAILED DESCRIPTION

Reference will now be made in detail to embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps. Furthermore, where the description of one or more embodiments uses the term “comprising,” those skilled in the art would understand that, in some specific instances, the embodiment or embodiments can be alternatively described using the language “consisting essentially of” and/or “consisting of.” As used herein, the term “about” means at most plus or minus 10% of the numerical value of the number with which it is being used.

In one aspect, the invention provides a device capable of detecting the differences in the mechanical properties of underlying tissues, which predicts the location of one or more anatomical features in a subject. In some embodiments, the one or more anatomical features can be selected from cartilage, tendon, bone, muscle, blood vessels, and vocal cords.

In some embodiments, the invention is comprised of three functional components: capacitive sensors, the dumbbell, and a Micro Linear Actuator with Limit Switches. These components directly contribute to obtaining the measurements leading to the force vs. displacement plot. The sensors may be combined with additional components, such as an I2C board and a controller board, that allow it to transfer that data into a computer with user interface.

In some embodiments, there are also several structural components that can be useful to the construct of the device. The dumbbell shell is where the dumbbell and the sensors are housed, the cap is what allows the dumbbell to be replaced, the rod is what allows the device to reach the vocal folds, and the handle stores the linear actuator as well as allows the user to maneuver the device easily.

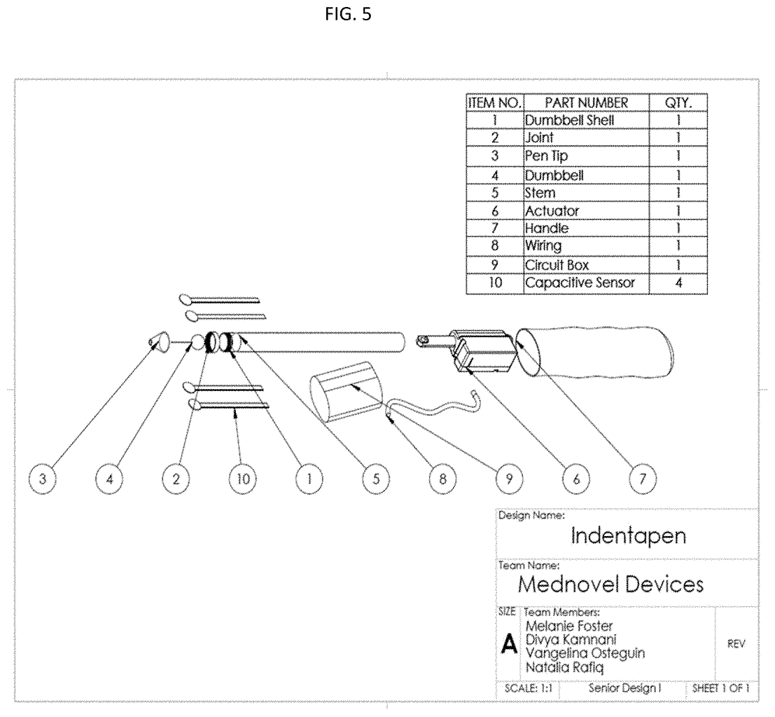

FIG. 5 shows an exploded view of one embodiment of the present invention. Dumbbell shell 1 encloses dumbbell 4 and interfaces with pen tip 3 through intervening joint 2. Stem 5 serves to house both dumbbell 4 and actuator 6. Handle 7 has a larger diameter than does stem 5 and encloses actuator 6, which may optionally be connected to a power supply via wiring 8. Wiring 8 may also serve to connect actuator 6 and/or capacitive sensor(s) 10 to circuit box 9. Circuit box 9 can then be connected to a digital display, computer, or graphical user interface (not shown) for display of one or more tissue stiffness determinations to a user.

Materials for construction of the inventive device are not particularly limiting. Any rigid, durable and inert metal, alloy, or plastic can be suitable. In some embodiments, dumbbell shell 1, joint 2, pen tip 3, dumbbell 4, stem 5, and handle 7 can be constructed of stainless steel or some equivalent or similar material.

Dimensions for the inventive device are not limiting. Most advantageously, given that a typical laryngoscope has an inner diameter of about 2 cm, the inventive device should have a diameter of 1 cm or less to allow it to fit inside the laryngoscope with enough room for the user to be able to move around and see the device.

Some embodiments designed for use in a human subject to measure the stiffness of the vocal folds can have the following design characteristics:
1. Measure mechanical properties
   The mechanical property that will be measured through this device is tissue stiffness. This will be accomplished by having a known and controlled displacement and measuring the varying force through the sensors. This data will be graphed in a force vs. displacement curve. The linear region of that curve will be calculated and represented as the stiffness.
2. In vivo usage
   In vivo usage will be accomplished by two means: 1) making the device small enough to fit in the laryngoscope and 2) making the device safe to use in the larynx. The device will be made safe by having a controlled and minimal displacement. To elaborate, other than possible user error, the device will not exert an excess force or displacement that would result in tearing or permanent indentation of the tissue. Its small size will also allow for the device to fit inside a laryngoscope and reach the patient's vocal folds.
3. Small in size
   The device is heavily restricted in dimensions. A laryngoscope has a diameter of 2 cm. This device has a diameter of 1 cm, thus being able to fit inside the laryngoscope with enough room for the user to be able to move around and see the device. The length of the device will be 26 cm. This is long enough to reach the vocal fold as well as give the physician enough room to reach it while looking through a microscope.
4. Flexible
   The device is able to fit inside a laryngoscope and measure various areas of the vocal fold. Additionally, there is extra space inside the laryngoscope for the device to be adjusted accordingly after the device is inserted inside the laryngoscope.
5. Durable
   The material that is being used for this device is stainless steel. The dumbbell, the cap, the rod, and the dumbbell house will all be made out of stainless steel. This material is a very high performance with good mechanical strength and high durability. This material will also act as a protector for the circuitry inside the rod.
6. Simple Interface
   The device has three simple steps for usage. First, the device is inserted through a laryngoscope and positioned accordingly to where the measurement is desired. Second, the user must press a button at the top of the handle where an actuator induces a known displacement to the device. Lastly, the user must interpret the measured and calculated data through a computer interface.
7. Portable
   The device will weigh less than 3 lbs.

Currently ex vivo studies for understanding mechanical properties of the vocal fold are done through nanoindentation. Nanoindentation can either be force controlled or displacement controlled in which one of the two variables is known and the other variable is measured. This technique has been used to measure the stiffness of a material by examining the linear region of the load vs. displacement graph.

While conventional nanoindentation machines are quite bulky and only used in ex vivo, the device of the present invention, Indentapen, aims to condense the machine into a compact pen that can be used in vivo. In this design, there is a small misshapen dumbbell tip acting as the indenter tip that is housed at the end of a long rigid tube. A linear actuator can be in use to set a constant distance during each test and capacitive sensors will be touching the dumbell to measure the force of the tissue resisting movement.

The dumbbell can have two spheres at its ends; a large one touching the capacitive force sensors placed within the tube and a much smaller one free to be in contact with the tissue.

The displacement induced can be done through the Actuonix PQ12-S Micro Linear Actuator with Limit Switches (Actuonix Motion Devices, Inc., Saanichton, BC, Canada).

As for the force, this can be measured through four Single Tact 8 mm 1 N/0.2 lb capacitive sensors. Three sensors can be placed equidistant from each other on the sides of the dumbbell while the fourth sensor can be placed at the top. Placement of the sensors can be such that they define a resultant force vector in 3D space. This vector would coincided with the displacement the tissue undergoes.

Since the displacement is known and the real force has been measured, a force vs. displacement graph can be plotted and from the linear region of this graph, the stiffness of the vocal fold is measured and displayed to the user.

The dimensions of each component were meticulously selected to adhere to patient and user convenience as well as the chosen parameters. In one embodiment, the dimensions of the linear actuator and the sensor are both fixed because they are pre manufactured components that can be purchased.

In one embodiment, the sensors have a diameter of 8 mm Considering that the three of the sensors will be placed consecutively, the device will occupy a minimum diameter of 24 mm The device is cylindrical, which means that 24 mm is the minimum inner circumference of the dumbbell house. Using the circumference=2 πradius equation, the minimum inner radius to house the sensors must be 7.6 mm The final radius of the shell also needs to account for the thickness of the sensor, 0.35 mm, and the double sided tape that will be used to secure the sensor to the inner dumbbell shell, 0.35 mm The shell will have a thickness of 0.5 mm Taking into account these additional parameters, the final inner diameter of the dumbbell house is 9 mm and the outer diameter is 10 mm

In this embodiment, there is an additional fourth sensor at the top of the dumbbell house which has the same 8 mm diameter. The slots at the top of the dumbbell house are large enough for the 3.5 mm×0.27 mm sensor tail to pass through and spaced in way that they do not interfere with the sensor placed at the top of the dumbbell house.

The dumbbell itself has two spheres, a larger one that will be in contact with the sensors and a smaller one that will be in contact with the vocal fold. The larger sphere has a diameter of 7.6 mm which is large enough to touch all the sensors and the small sphere has a diameter of 0.5 mm While traditional nanoindentation machines have tips that are 0.1 mm in diameter, this design opted to have a larger diameter so avoid instability which may result due to the shaft if the dimensions are too thing. The shaft connecting these two spheres has a thickness of 0.56 mm and length of 1.5 mm These dimensions were decided keeping in mind the shorter the shaft, the more stable the device. The dumbbell component is interchangeable for sterilization purposes which means that it needs to be easily removable. This is the purpose of the cap. The cap needs to be able screw on and off easily, which in turn affected the length of the shaft. The cap is 1 cm long, which makes removable and reassembly easy for the user.

Another key component is the rod. It is designed to be 12 cm long. Considering that the distance from the mouth to the vocal fold is about 7 cm, this should be long enough to reach most patients' vocal folds. The handle is 10.3 cm long and 3 cm wide. Most comfortable handles are well within these dimensions, ensuring that it will be comfortable for most users. It is also wide enough to fit the linear actuator inside it.

While the invention has been described with reference to certain particular examples and embodiments herein, those skilled in the art will appreciate that various examples and embodiments can be combined for the purpose of complying with all relevant patent laws (e.g., methods described in specific examples can be used to describe particular aspects of the invention and its operation even though such are not explicitly set forth in reference thereto).

### EXAMPLES

All patents and publications mentioned and/or cited in this specification are indicative of the level of those skilled in the art to which the invention pertains. All patents and publications mentioned and/or cited herein are incorporated by reference to the same extent as if each individual publication was specifically and individually indicated as having been incorporated by reference in its entirety.

### Components

The indentapen has three key functional components: the Single Tact 8 mm 1N/0.2 lb capacitive sensors, the dumbbell, and the Actuonix PQ12-S Micro Linear Actuator with Limit Switches. These components directly contribute to obtaining the measurements for the force vs. displacement graph. The sensors come with additional components, an I2C board and an Arduino board, that allow it to transfer that data into a computer interface.

There are also several structural components that are essential to the construct of the device. The dumbbell house is where the dumbbell and the sensors are housed, the cap is what allows the dumbbell to be replaced, the rod is what allows the device to reach the vocal folds, and the handle stores the linear actuator as well as allows the user to maneuver the device easily.

1. Sensors: The sensors that will be used in the devices are the Single Tact 8 mm 1N/0.2 lb capacitive sensors. When the smaller sphere of the dumbbell comes into contact with the vocal fold, the larger sphere feels the force and transfers it to the capacitive sensors. All four of the sensors are attached to an I2C board, which is connected to the Arduino board.

2. Arduino Board: The arduino board is intermediate between the sensors and the computer. It is able to transfer the data and quantify it onto a screen.

3. Dumbbell: The dumbbell is the interface that will come into contact with the vocal folds and the sensors. It consists of two spheres, a large and a small one, joined by a short shaft. The large sphere exerts the force felt onto the sensors and the small sphere comes into contact with the vocal fold.

4. Linear Actuator: Actuonix PQ12-S Micro Linear Actuator with Limit Switches will be the linear actuator used in this device. It will be stored inside the handle and be able to displace a distance of 1 mm.

5. Dumbbell House: This components houses the sensors and the dumbbell. Three of the sensors will be placed on the inner sides of the dumbbell house. They will be equally spaced apart. The fourth sensor will be placed at the top of the dumbbell house.

6. Cap: The cap serves to be able to easily remove the dumbbell from its house and replace it for sterilization purposes. It is screwed on and off easily.

7. Rod: The is hollow and in addition to serving as means to reach the vocal fold, it also houses the additional wiring from the sensors.

8. Handle: The handle is modeled after an ice cream scoop handle. It is also hollow and large enough to house the linear actuator while being easy to hold for the user.

### Discussion of Functionality

Equations used to evaluate this design:

### 1. Equations for calculating Stiffness

K=ΔF/Δ1

ΔF—Change in force

A1—Change in distance

The distance displaced is known through the linear actuator and the force is calculated through the sensors. Given these variables, a force vs. displacement plot can be made in which the stiffness can be calculated by examining the slope of linear region of the graph.

### 2. Equations to Convert Capacitance to Force

C=0rAd

0andr—Properties of the dielectric material between the plates
A—Area of the plates
d—Distance between the plates

The dielectric must be compressible so that an external force can be applied to the capacitor and the change in d will result in a change of capacitance.

Δd=Fd0EA

Δd—change in distance

F—Applied force

E—Elastic modulus of the dielectric (E=1.4 kPa)

A—Area of the plates (A=5.026×10⁻⁵ mm²)

d₀—Initial distance between the plates (d₀=0.35 mm)

Hooke's law states that the applied force is directly related to the displacement of the plates, therefore changes in capacitance enable the applied force to be computed.

Rearranging the capacitance equation to focus on the changed capacitance yield the below equation that shows a relationship between capacitance and applied force:

Cf=0rAd (1−FAE)

Rearranging once again, we see how we can acquire force from knowing the change of capacitance and the final capacitance:

F=EAΔCCf

Note that “final capacitance” (C_{f}) doesn't mean the last capacitance reading but could be any capacitance that is not the initial capacitance value.

### 3. Equations to Find True Force

$$[ \overline{{F}_{R}} ] = \sqrt{\sum {F}_{x}^{2} + \sum {F}_{y}^{2} + \sum {F}_{z}^{2}}$$

$${F}_{x} - Force in the x direction$$

$${F}_{y} - Force in the y direction$$

$${F}_{z} - Force in the z direction$$

Each of the forces, Fₓ , F_{y} , and F_{z}, are obtained from the three sensors touching the sides of the dumbbell. The way the force is calculated here can be described as defining a vector in the 3D space. First, the magnitude (F_{R}) must be calculated. Next, the angle at which the smaller end of the dumbbell is hitting the vocal fold must also be taken into account. This can be done with the equations below.

$$α = {cos}^{- 1} \frac{\text{?}}{[ \text{?} ]}$$

$$β = {cos}^{- 1} \frac{\text{?}}{[ \text{?} ]}$$

$$γ = {cos}^{- 1} \frac{\text{?}}{[ \text{?} ]}$$

$$\text{?} \text{indicates text missing or illegible when filed}$$

The most important angle in question is γ because it is the angle used for force correction. Nanoindentation works ex vivo because it always is at 90 degrees. When performing in vivo, that may not always be the case. Thus this equation must be used to find the true force exerted by the vocal fold:

F=F′ sin θ

F—true force

F′—Force obtained through fourth sensor at the top of the dumbbell

Θ—γ calculated from previous equations

### 3. Equations to Find Displacement Induced

D=rt

D—distance

R—rate

T—time

The linear actuator that will be used in this setting can be adj

### Discussion of Specifications

The functional requirements of this device can be broken down into seven specifications. This framework was obtained through a tedious interview process of various clinicians, speech pathologist and residents that are knowledgeable in the vocal fold field. The parameters are as follows:

### 1. Measurement of Mechanical Properties

The ultimate goal of this device is to be able to measure the mechanical properties of the vocal fold. Mechanical properties entails parameters such as the Young's Modulus, storage modulus, elasticity, or tissue density. In this device, we have elected to pursue measuring the stiffness of the vocal fold.

### 2. In Vivo Usage

While measurement of the mechanical properties of the vocal fold is possible and has been done ex vivo, a major component that begets uniqueness in this device is its ability to be used in vivo. This device should be designed to be able to fit inside a laryngoscope and be safe to use on the soft tissue.

### 3. Small in Size

Area of interest is the vocal fold, limiting the mode of entrance for any device to be done orally. Additionally, clinicians make use of a laryngoscope that has width of 2 cm, restricting the dimensions of the device. With this in mind, the device should be less than 2 cm in width and length should be less than 40 cm. The distance between the patient's mouth and the physician's microscope is approximately 40 cm. Therefore, the device needs to be able to fit in between that space for the physician to comfortably use it.

### 4. Flexible

The distance from the mouth to the laryngoscope is approximately 7 cm. The device should be flexible enough to move about and measure different areas of the vocal fold as well as fit inside the laryngoscope.

### 5. Durable

We intend for this device to be used for at least a year before replacement. This will be accomplished by using high quality materials that do not fracture or malfunction in long term use.

### 6. Simple Interface

Currently, ENTs who treat vocal folds are highly trained and experienced. However, not all ENTs are specialized in vocal folds and cannot rely on audio or visual examination to treat patients. This device aims to allow vocal fold treatment to be more universal amongst ENTs regardless of his or her specialization. Therefore, it must be easy to use and interpret. This device addresses this by having three simple steps for usage. It also has an easy grip handle to give the user more comfort.

### 7. Portable

As mentioned previously, current nanoindentation devices are quite bulky. By making this device small, we are also making it portable. The device should have a weight of less than 3 lbs., thus making it easy to hold, carry, and move around.

| Functional | |
|---|---|
| Specifications | Quantification |
| Size | Yes; the diameter is 1.2 cm and able to fit in a |
| (diameter) | laryngoscope |
| Flexible | No; the device will not bend, but is flexible in the |
| | sense that it can measure different parts of the |
| | vocal fold; the device is also adjustable in that |
| | the distance displaced can be changed |
| Durable | Yes; all the materials used in the device are strong |
| Portable | Yes |
| Measure | Yes; the device is able to measure the stiffness |
| mechanical | of the vocal fold |
| properties | |
| Simple | Yes |
| interface | |
| In vivo | Yes |
| usage | |

### Strengths & Weaknesses

### i. Strengths

Uses a proven concept of measuring vocal fold properties

Can measure multiple spots on vocal fold

Simple in usage and functionality

### ii. Weaknesses

Measurement in horizontal direction is not as sensitive as measurement in vertical direction

Would be most effective in larger laryngoscopes, thus may not be suitable for children

### Sketch/Drawing

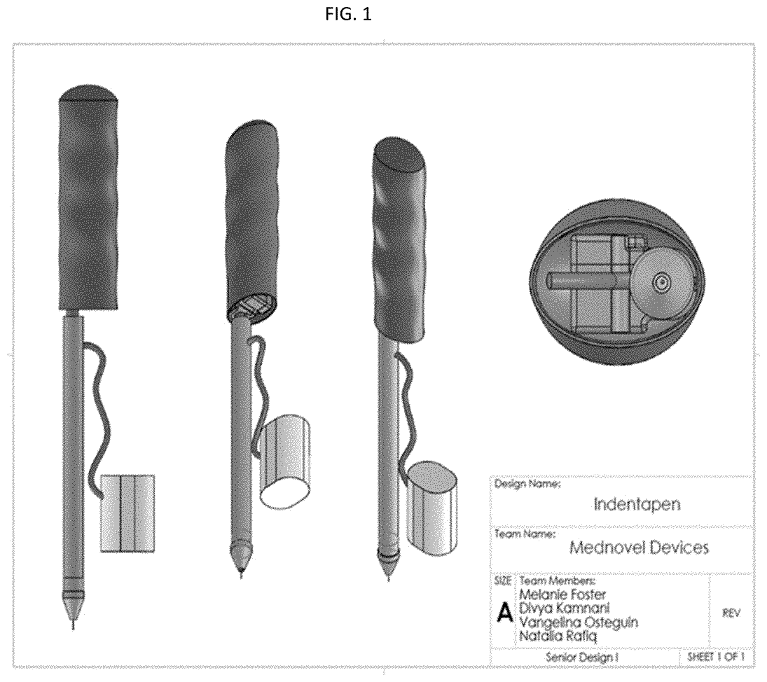

FIG. 1 shows the whole nanoindentation pen device.

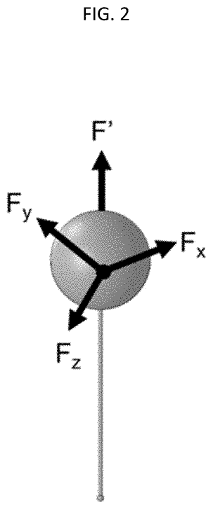

FIG. 2 shows the four forces that are acting upon the dumbbell.

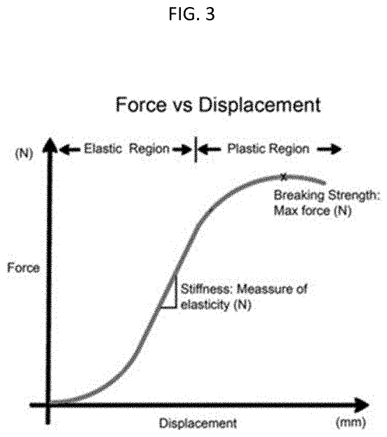

FIG. 3 shows the Force vs. Displacement curve.

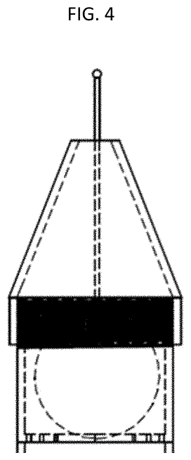

FIG. 4 shows dumbbell resting inside dumbbell house.

In conclusion, the nanoindentation pen uses basic concepts of statics and mechanics to calculate the stiffness of the vocal fold based on a known displacement and a measured force. The pen itself is able to reach most sides of the vocal fold, making it ideal for all types of treatments. The device can be used to measure the vocal folds before and after treatments and provide some quantitative data on the mechanical properties. Despite its setbacks, the nanoindentation pen is very practical from both a clinical and engineering standpoint.

What is claimed is:

1. A device for measurement of mechanical properties of a tissue in a subject, the device comprising:
a dumbbell comprising an elongated shaft, the shaft being affixed at a first end to a pen tip for contacting the tissue, the shaft being affixed at a second opposite end to a terminal member;
a linear actuator; and
one or more pressure sensors that are in physical communication with the terminal member, the pressure sensor(s) generating a signal that indicates a force exerted on the terminal member by the pen tip via the shaft.

2. The device of claim 1, wherein the linear actuator can be configured to reliably and repeatedly move the shaft axially over a constant distance.

3. The device of claim 1, wherein the pen tip is conical in shape, an apex of the cone being oriented to contact the tissue when the device is operated.

4. The device of claim 1, wherein one or both of the pen tip and the terminal member are spherical.

5. The device of claim 4, wherein a radius of the terminal member is larger than a radius of the pen tip.

6. The device of claim 5, wherein the shaft has a cylindrical shape.

7. The device of claim 1, wherein the pressure sensor(s) is/are capacitive pressure sensors.

8. The device of claim 7, comprising four pressure sensors, wherein three pressure sensors are distributed at regular intervals radially about the shaft and one pressure sensor is in line with an axis of the shaft.

9. The device of claim 1, wherein the mechanical properties to be measured include tissue stiffness.

10. The device of claim 1, wherein the signal is an electrical signal.

11. The device of claim 10, further comprising circuitry for calculating a tissue stiffness from the electrical signal.

12. The device of claim 1, further comprising a dumbbell shell that encloses all or part of the dumbbell.

13. The device of claim 1, wherein the subject is a human subject.

14. The device of claim 13, wherein the human tissue is vocal folds.

15. The device of claim 1, wherein a length of the device is about 26 cm.

16. The device of claim 1, wherein the device is cylindrical in shape overall and has a diameter of 1 cm or less.

17. The device of claim 1, wherein the device is used in vivo.

18. The device of claim 1, wherein the device is used ex vivo.

19. The device of claim 1, wherein the pen tip, the terminal member and the shaft are all formed of stainless steel.

20. The device of claim 1, wherein the pen tip, the terminal member and the shaft are continuous.

21. The device of claim 1, wherein the device is portable.

22. The device of claim 1, wherein the device has a total weight of less than three pounds.

23. The device of claim 1, further comprising a display that displays a tissue stiffness quantity to a user.

24. A method for measuring stiffness of a biological tissue, the method comprising:
providing a dumbbell, the dumbbell having an elongated shape and comprising a pen tip at a first end, a terminal member at a second end and a shaft connecting the pen tip and the terminal member;
contacting the pen tip with the biological tissue;
actuating a linear actuator such that the pen tip displaces a portion of the biological tissue; and
measuring a force exerted on the terminal member by the biological tissue via the pen tip and the shaft.

25. The method of claim 24, wherein the biological tissue is human biological tissue.

26. The method of claim 25, wherein the human biological tissue is vocal folds.

27. The method of claim 26, wherein the method is performed in vivo.
